(19) United States
(12) Patent Application Publication
(10) Pub. No.: US 2025/0333694 A1
(43) Pub. Date: Oct. 30, 2025

(54) Methods of Making Oligodendrocyte Progenitor Cells

(71) Applicant: BLUEROCK THERAPEUTICS LP, Cambridge, MA (US)

(72) Inventors: Lev STARIKOV, New York, NY (US); Juliana HSU, New York, NY (US); Thomas Cutia, New York, NY (US); Mark Ebel, New York, NY (US); Mark Tomishima, New York, NY (US)

(21) Appl. No.: 18/868,319

(22) PCT Filed: May 4, 2023
(86) PCT No.: PCT/US2023/020916

§ 371 Date: Nov. 22, 2024

Related U.S. Application Data
(60) Provisional application No. 63/345,251, filed on May 24, 2022.
(60) Provisional application No. 63/356,536, filed on Jun. 29, 2022.

(51) Int. Cl. C12N 5/079; A61K 35/30; A61P 25/00; C12M 1/02

(52) U.S. Cl. CPC C12N 5/0622; A61K 35/30; A61P 25/00; C12M 27/00

(57) ABSTRACT

The present disclosure is directed to methods of producing oligodendrocyte progenitor cells (OPCs). In addition, methods of treating demyelinating diseases using oligodendrocyte progenitor cells (OPCs) are also disclosed.

### CROSS REFERENCE TO RELATED APPLICATIONS

This application is a 371 of International Application No. PCT/US23/20916, filed May 4, 2023, which claims the benefit of U.S. Provisional Patent Application No. 63/345,251, filed May 24, 2022 and U.S. Provisional Patent Application No. 63/356,536, filed Jun. 29, 2022, the contents of which are incorporated herein by reference in its entirety.

### BACKGROUND

Oligodendrocyte progenitor cells (OPCs) are resident glial cells in the central nervous system (CNS) that are highly mobile and readily differentiate into axon-wrapping mature oligodendrocytes throughout lifetime. Emerging cell replacement therapies leverage OPCs in treating demyelinating conditions in which endogenous OPCs are dysfunctional. The myelin sheath is an important structure of the nervous system, and demyelinating diseases can cause cognitive, memory, and motor dysfunctions that can seriously affect a patient's quality of life. Derivation of OPCs from induced pluripotent stem cells (iPSCs) provides a promising platform with allogenic capability. However, major obstacles remain in transforming production of oligodendrocyte lineage cells from petri-dish to the clinical scale (estimated >10⁸ cells per demyelinated lesion), and a prolonged timeframe compared to most neuronal differentiations.

### SUMMARY OF THE DISCLOSURE

In one aspect, the disclosure provides a method of producing oligodendrocyte progenitor cells (OPCs) comprising obtaining induced pluripotent stem cells (iPSCs) from a subject; differentiating the iPSCs into neural progenitor cells (NPCs); culturing the NPCs in a suspension culture in a bioreactor with agitation to form oligospheres; dissociating the oligospheres using an enzymatic treatment and mechanical agitation into single oligodendrocyte progenitor cells (OPCs); and cryopreserving the single OPCs in a step-wise freezing process.

In some embodiments, the bioreactor is an impeller-driven DASbox mini-bioreactor. In some embodiments, the DASbox mini-bioreactor has a speed of rotation of 100 rpm (rotation per minute) for about 1 hour followed by 400 rpm for about 2 hours.

In some embodiments, the agitation by the impeller-driven DASbox mini-bioreactor leads to high post-thaw cell viability after cryopreservation.

In some embodiments, the enzymatic treatment comprises 1× AccuMax and 2× TrypLE Select diluted in HBSS or 4× TrypLE Select diluted in HBSS (Hanks' Balanced Salt Solution). In some embodiments, the enzymatic treatment does not include exogenous DNase I addition.

In some embodiments, the NPCs are differentiated in the suspension culture for about 60 days. In some embodiments, the OPCs express lineage markers CD9, O4, SOX10, OLIG2, and NKX2.2 by about Day 60. In some embodiments, the NPCs are differentiated in the suspension culture for about 20 days, about 25 days, about 30 days, about 35 days, about 40 days, about 45 days, about 50 days, about 55 days, about 60 days, about 65 days, about 70 days, about 75 days, about 80 days, about 85 days, about 90 days, about 95 days, about 100 days, about 105 days, about 110 days, about 115 day or about 120 days.

In another aspect, this disclosure provides a method of treating a demyelinating disease or disorders comprising administering oligodendrocyte progenitor cells (OPCs) into a central nervous system of a subject to be treated allowing the administered oligodendrocyte progenitor cells (OPCs) to engraft in the central nervous system and thereby restore the functions supported by OPCs as well as mature oligodendrocytes. In another aspect this disclosure provides a method of treating a demyelinating disease comprising administering oligodendrocyte progenitor cells (OPCs) into a central nervous system of a subject to be treated allowing the administered oligodendrocyte progenitor cells (OPCs) to engraft in the central nervous system thereby restoring expression of myelin basic protein (MBP).

In some embodiments, a scalable differentiation platform produces functional oligodendrocyte progenitor cells (OPCs), wherein the OPCs are produced in about 60 days in vitro. In some embodiments, the OPCs are differentiated from neural progenitor cells (NPCs).

In some embodiments, in the scalable differentiation platform, the NPCs are differentiated in a suspension culture to form oligospheres. In some embodiments, the suspension culture is in an impeller-driven mini-bioreactors.

In some embodiments, the oligospheres generated in the impeller-driven mini-bioreactors allow for about 4-fold, about a 5-fold, about a 6-fold, about a 7-fold, about an 8-fold, about a 9-fold and about a 10-fold expansion in cell yield and a physical uniformity of spheres.

In some embodiments, the OPCs express lineage markers CD9, O4, SOX10, OLIG2, and NKX2.2 by about Day 60.

In some embodiments, a lactate dehydrogenase release serves as a cell health indicator during sphere dissociation.

In one aspect, this disclosure provides an oligodendrocyte progenitor cell (OPC) produced by a method comprising obtaining induced pluripotent stem cells (iPSCs) from a subject, differentiating the iPSCs into neural progenitor cells (NPCs), culturing the NPCs in a suspension culture in a bioreactor with agitation to form oligospheres, dissociating the oligospheres using an enzymatic treatment and mechanical agitation into single oligodendrocyte progenitor cells (OPCs), and cryopreserving the single OPCs in a step-wise freezing process.

In another aspect, this disclosure provides oligodendrocyte progenitor cells (OPCs), wherein the OPCs express lineage markers CD9, O4, SOX10, OLIG2, and NKX2.2 by about Day 60 after in vitro differentiation of neural progenitor cells (NPCs) in a suspension culture in a bioreactor.

In another aspect, this disclosure provides oligodendrocyte progenitor cells (OPCs), wherein the OPCs are modulated by WNT.

In some embodiments, the oligodendrocyte progenitor cells (OPCs) are SOX10 and O4 positive at about Day 60.

In some embodiments, the oligodendrocyte progenitor cells (OPCs) the percentages of SOX10 and O4 positive cells at Day 60 increase to >50% with an addition of WNT modulator between about Day 40 to about Day 60.

### BRIEF DESCRIPTION OF THE FIGURES

The patent or application file contains drawings executed in color. Copies of this patent or patent application publication with color drawing(s) will be provided by the Office upon request and payment of the necessary fee.

FIG. 1A-1E. (A) A diagram depicting the protocol by which oligodendrocyte progenitor cells are differentiated from human induced pluripotent stem cells (iPSCs). (B) Lactate concentrations in the spent media are measured daily during adherent culture phase. Data shown are from multiple representative runs. (C) At day 12, adherent cells, namely neural progenitor cells (NPCs), are dissociated enzymatically prior to seeding into suspension culture. The cell yield (millions/cm²) are plotted against vessel types. CS, CellStack of surface area of 636 cm². (D) The slopes of lactate curve from Day 0-12 are plotted against total cell yield at Day 12 and show positive correlation. (E) Correlations are found among seeding density at Day 0, slope of lactate curve, and total cell yield at Day 12.

FIG. 2. Monolayer cells at differentiation Day 12 were characterized using flow cytometry, RT-qPCR, and immunofluorescence for the expression of characteristic neural progenitor markers. Day 12 cells express the ventralization markers OLIG2 and NKX2.2 by flow and RT-qPCR. HOXB4 expression indicates anterior spinal cord specification. Scale bars are 100 μm.

FIG. 3A-3B. (A) Suspension cells were cultured either in stationary vessels or agitation format in a bioreactor. (B) Agitation generated more uniform sphere size and shape. Flow analysis showed SOX9+/NKX2-2+ double expression. Immunofluorescence staining of sphere sections at Day 20 (D20) showed differences in sphere organization shown by SOX9 and NKX2.2 expression. Spheres dissociated at Day 60 (D60) are stained for O4 by flow and IF. Stationary and agitation spheres showed similar gene expression patterns for neural and glial progenitor markers as assayed by RT-qPCR panel. Scale bars, 200 μm in bright field images and 100 μm in fluorescent images.

FIG. 4A-4B. The % of OPCs committed to oligodendrocyte fate at Day 60 is regulated by WNT (Wingless/Integrated) signaling. The co-expression of SOX10 and O4 antigen signify the fate commitment of OPC to oligodendrocyte lineage. The percentages of SOX10 and O4 positive cells at Day 60 increase to >50% with the addition of WNT modulator between Day 40 to Day 60 (B) compared to those without any manipulation (A). WNT modulator (agonist) added at 3 uM.

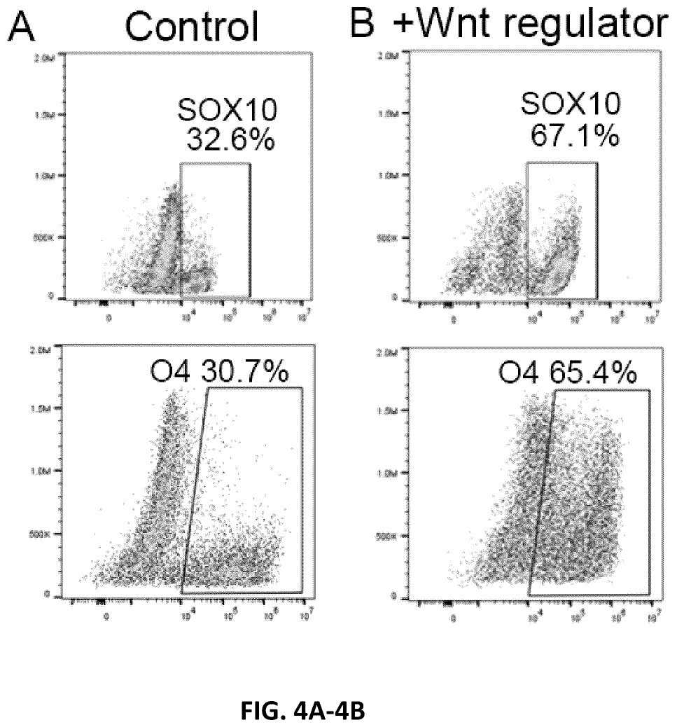

FIG. 5. Dissociation and cryopreservation of oligodendrocyte progenitor cell (OPC) cell product. Spheres were enzymatically dissociated into a single cell suspension by passing through a 70 μm strainer. Dissociation progress was monitored by tracking cell concentration over time, cell health was simultaneously monitored by lactate dehydrogenase (LDH) release as an indicator of cell death. Data shown are technical replicates from one representative experiment. Single cells were cryopreserved using a control rate freezing program and transferred to liquid nitrogen long term storage. Agitation bioprocess supports higher seeding density than stationary culture and translates to significantly higher cell yield by Day 60. Upon thaw, oligodendrocyte progenitor cell (OPC) cell product was comparable between both stationary and agitation workflows. Individual data points in bar graphs represent 2-3 separate experiments with 2-3 technical replicates per experiment.

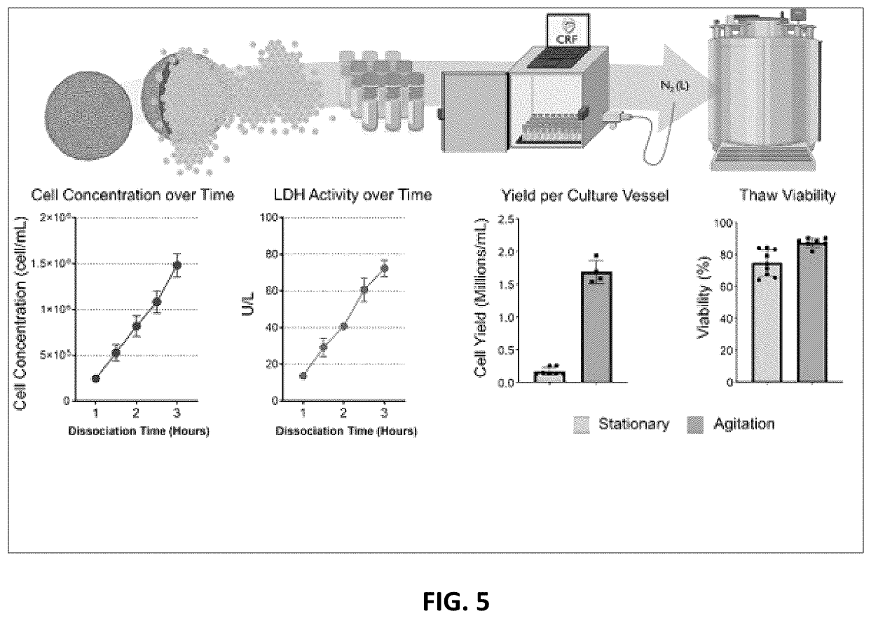

FIG. 6. Comparison of the single cell transcriptome of stationary and agitation spheres at Day 20 of culture. Unbiased single-cell RNA-sequencing showed that both culture methods induce NKX2-2 ventral fated neuronal (MNX1) and glial progenitor (SOX9) populations.

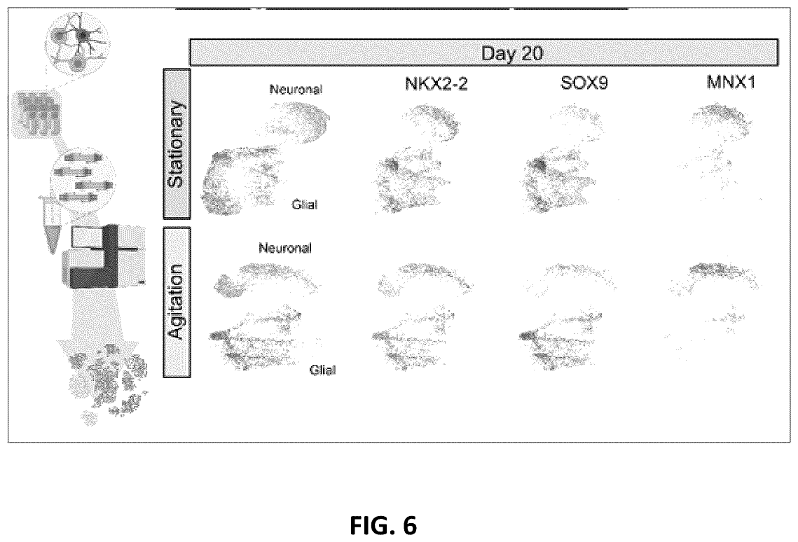

FIGS. 7A-7C. Day 60 dissociated cells from stationary culture were transplanted into P13 MBP (myelin basic protein) deficient shiverer mice (Rag2/ll2 KO) in an immune-compromised genetic background. (B) Schematic of OPC injection. (C) Representative images of OPCs integrated in the corpus callosum (left) and cerebellum (right). Human cells express STEM121 (red), and mature oligodendrocytes restore the expression of the myelin protein MBP (green). Scale bars are 200 μm.

FIG. 8. Comparison of the effect of OPCs vs. vehicle in ataxic mice. OPCs rescued ataxic gait compared to vehicle controls.

### DETAILED DESCRIPTION OF THE DISCLOSURE

### Definitions

Unless defined otherwise, all technical and scientific terms used herein have the same meaning as commonly understood by one of ordinary skill in the art to which this invention belongs.

As used herein, the term “about” refers to an approximately +/−10% variation from a given value.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The term “pluripotent stem cells” or “PSCs,” as used herein, has its usual meaning in the art, i.e., self-replicating cells that have the ability to develop into endoderm, ectoderm, and mesoderm cells. In some embodiments PSCs are human PSCs. PSCs include embryonic stem cells (ESCs) and induced pluripotent stem cells (“iPS cells” or “iPSCs”). The terms ES cells and iPS cells have their usual meaning in the art.

Terms such as “treating” or “treatment” or “to treat,” as used herein, refer to therapeutic measures that cure, restore regenerative function, slow down, lessen symptoms of, and/or halt progression of a diagnosed pathologic disease or disorder. Thus, those in need of treatment include those already with the disorder. In certain embodiments, a subject is successfully “treated” for a disease or disorder if the subject shows, e.g., total, partial, permanent, or transient, alleviation or elimination of any symptom associated with the disease or disorder.

As used herein, the phrase “administering” refers to the physical introduction of an agent to a subject, using any of the various methods and delivery systems known to those skilled in the art. Exemplary routes of administration for the oligodendrocyte progenitor cells (OPCs) prepared by the methods disclosed herein include intravenous, intramuscular, subcutaneous, intraperitoneal, spinal or other parenteral routes of administration, for example by injection or infusion. The “phrase parenteral routes of administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Non-parenteral routes include an oral, topical, epidermal or mucosal route of administration, for example, orally, intranasally, vaginally, rectally, sublingually or topically. Administering can also be performed, for example, once, a plurality of times, and/or over one or more extended periods.

The term “Oligodendrocyte Progenitor Cells (OPCs)” as used herein, refer to a subtype of glial cells responsible for myelin regeneration. OPCs represent a high proliferative cell population resident in the CNS (central nervous system) of adult mammals and humans. OPCs can be used as a treatment for spinal cord injury, stroke, Parkinson's disease, Multiple Sclerosis, Cerebral Palsy, as well as demyelination conditions including leukodystrophy (e.g. Krabbe disease, Canavan's disease), Neuritis Myelitis Optica spectrum (including NMO, TM) and radiation induced brain injury (RBI).

The term “neural progenitor cells (NPCs)” as used herein, refer to progenitor cells of the CNS that give rise to many, if not all, of the glial and neuronal cell types that populate the CNS. NPCs do not generate the non-neural cells that are also present in the CNS, such as immune system cells. NPCs can be generated in vitro by differentiating embryonic stem cells or induced pluripotent stem cells (iPSC).

The term “suspension culture” as used herein, refers to a type of cell culture in which single cells or small aggregates of cells are allowed to function and multiply in an agitated growth medium, thus forming a suspension.

The term “bioreactor” as used herein, refers to any manufactured device or system that supports a biologically active environment. Bioreactors are vessels or tanks in which whole cells or cell-free enzymes transform raw materials into biochemical products and/or less undesirable by-products. Bioreactors may be operated as batch reactors or continuously, aerobically or anaerobically, and with pure or mixed cultures. In some embodiments, the bioreactor is an impeller-driven DASbox mini-bioreactor. In other embodiments the bioreactor is a Thermo-mixer C or Miltenyi gentleMACS octo-dissociator.

The term “central nervous system” as used herein, refers to the spinal cord, brain, and cerebrospinal fluid (CSF).

The term “oligospheres” as used herein, refer to an aggregate of oligodendrocyte progenitor cells.

The term “enzyme” as used herein, refers to biological molecules (typically proteins) that significantly speed up the rate of virtually all of the chemical reactions without being consumed or permanently altered themselves. In some embodiments, an enzyme mixture acts as a dissociation agent and helps to dissociate oligospheres into single oligodendrocyte progenitor cells (OPCs).

The term “dissociation” as used herein, refers to a process in which a cell mass is separated into single cells. In some embodiments, the cell mass is an aggregate of oligodendrocyte progenitor cells (OPCs) or oligospheres.

The term “cryopreservation” as used herein, refers to a process that preserves organelles, cells, tissues, or any other biological constructs by cooling the samples to very low temperatures. In some embodiments, the cryopreservation is a step-wise freezing process.

The term “lineage markers” as used herein, refers to characteristic molecules for cell lineages, e.g., cell surface markers, mRNAs, microRNAs, or internal and secreted proteins. In some embodiments, oligodendrocyte progenitor cells (OPCs) express lineage markers CD9, O4, SOX10, OLIG2, and NKX2.2.

The term “demyelinating disease” or “demyelinating disorder” as used herein, refers to any condition that results in damage to the protective covering (myelin sheath) that surrounds nerve fibers in the brain, optic nerves and spinal cord. When the myelin sheath is damaged, nerve impulses slow or even stop, causing neurological problems. Examples of demyelinating diseases are Multiple Sclerosis, Parkinson's disease, Guillain-Barre Syndrome, Stroke etc.

The term “myelin basic protein (MBP)” as used herein, refers to the major protein component of myelin and is produced by oligodendrocytes. MBP is released into the extracellular matrix after shearing damage to white matter tracts (i.e., diffuse axonal injury).

The term “cells” as used herein, refers to the basic membrane-bound unit that contains the fundamental molecules of life and of which all living things are composed.

The term “a scalable differentiation platform” as used herein, refers to a process to differentiate neural progenitor cells (NPCs) in a suspension culture in a bioreactor with agitation to form oligospheres.

The term “a closed system” as used herein, refers to a system with equipment designed and operated such that the product is not exposed to the room environment.

The term “ataxia” as used herein, refers to a group of neurological conditions. There are several types of ataxia, including: ataxia telangiectasia (AT), episodic ataxia, Friedreich's ataxia, multiple system atrophy (MSA) and spinocerebellar ataxia. This condition occurs when the cerebellum is damaged. Ataxia herein also refers to a group of disorders that affect co-ordination, balance and speech. Any part of the body can be affected, but people with ataxia often have difficulties with balance and walking, speaking and/or swallowing.

### General Description

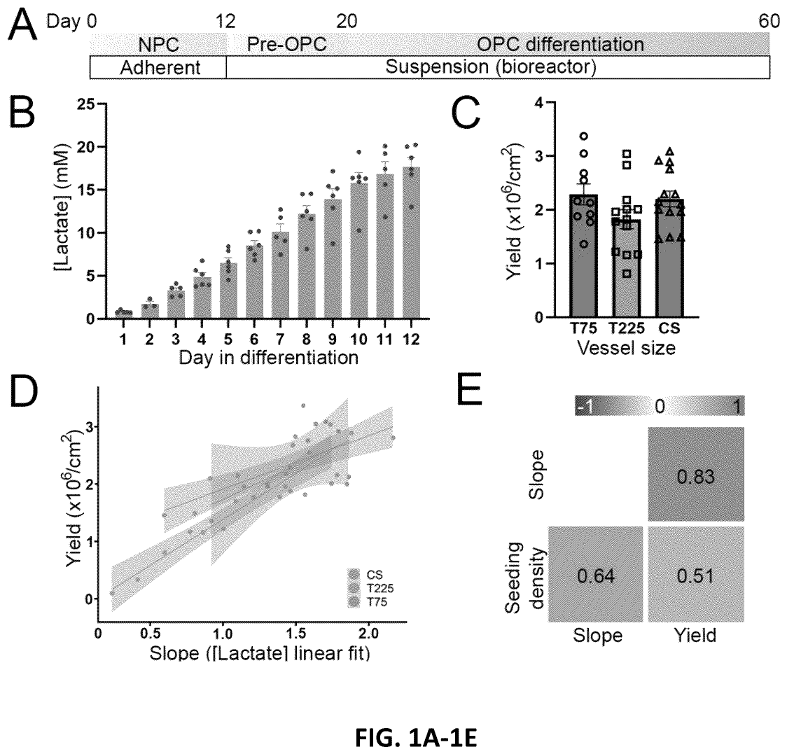

The present disclosure describes a method of producing oligodendrocyte progenitor cells (OPCs) using a scalable differentiation platform that enables the production of functional OPCs in about 60 days in vitro. FIG. 1 depicts the protocol by which oligodendrocyte progenitor cells are differentiated from human induced pluripotent stem cells (iPSCs). FIG. 1. also depicts (B) Lactate concentrations in the spent media which measured daily during adherent culture phase. Data shown are from multiple representative runs. (C) At day 12, adherent cells, namely neural progenitor cells (NPCs), are dissociated enzymatically prior to seeding into suspension culture. The cell yield (millions/cm²) are plotted against vessel types. CS, CellStack of surface area of 636 cm².

### Generation of Neural Progenitor Cells (NPCs) from Pluripotent Stem Cells

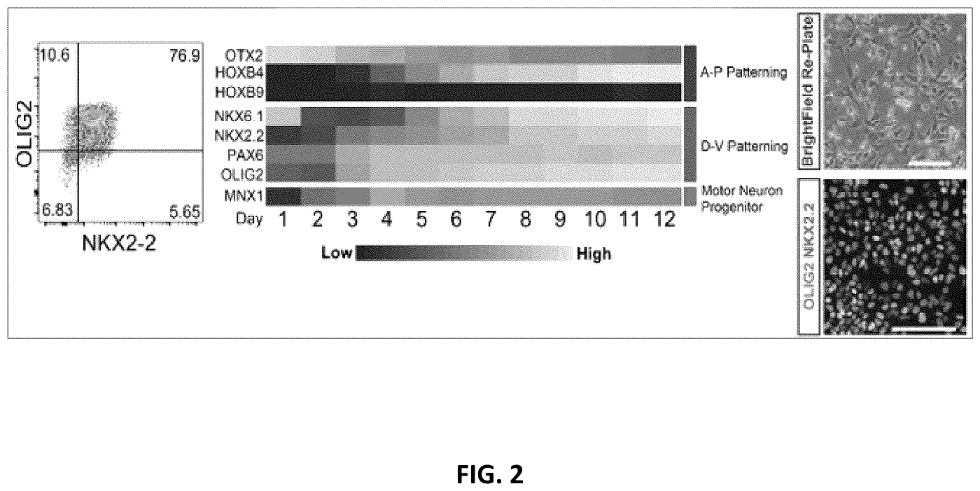

In some embodiments, the first phase of the disclosed process is to derive neural progenitor cells (NPCs) from pluripotent stem cells by a dual-SMAD inhibition method (Chambers S. M, Craft C. A, Papapetrou E. P, Tomishima M, Sadelain M, Studer L. Highly efficient neural conversion of human ES and iPS cells by dual inhibition of SMAD signaling. Nat Biotechnol. 2009;27(3):275-280. Epub 2009 Mar. 1. Erratum in: Nat Biotechnol. 2009 May;27(5):485.) which is a well-established method to derive neural progenitor cells from pluripotent stem cells. In accordance with the disclosure, the dual-SMAD inhibition method coupled with activation of sonic hedgehog signaling induced robust co-expression of OLIG2 and NKX2.2 in up to about 80% of neural progenitor cells (NPCs) at day 12 of monolayer differentiation process as shown in FIG. 2.

### Differentiation of Neural Progenitor Cells (NPCs) into Oligospheres

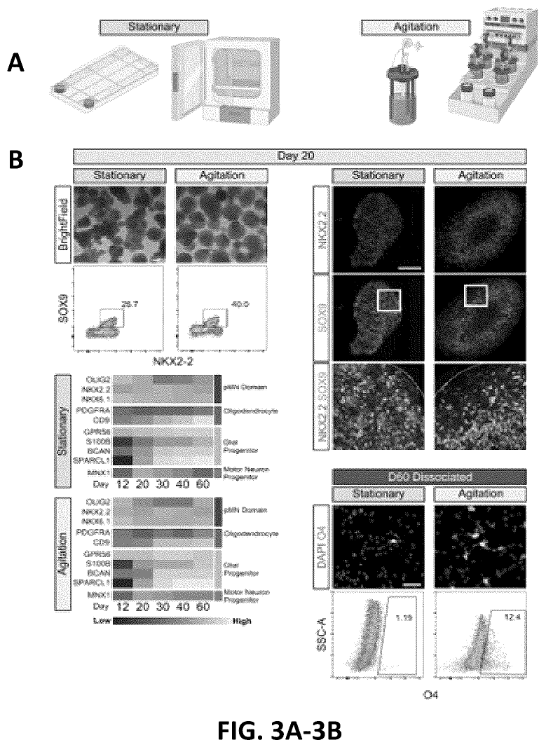

In some embodiments, these NPCs were then moved to suspension culture to form oligospheres in either stationary culture or impeller-driven mini-bioreactors for the remainder of the differentiation as shown in FIG. 3A. The oligospheres generated in bioreactors allowed for about a 4-fold expansion in cell yield and improved physical uniformity of spheres. Agitation generated more uniform sphere size and shape, and increased SOX9+/NKX2-2+ expression as shown by flow cytometry (FIG. 3B).

Immunofluorescence staining of sphere sections at Day 20 (D20) showed differences in sphere organization and SOX9 and NKX2.2 expression (FIG. 3B). By Day 60, OPCs expressed lineage markers CD9, O4, SOX10, OLIG2, and NKX2.2. Cells generated by stationary culture and bioreactors shared remarkable similarities in protein and gene expression. Stationary and agitation spheres showed similar gene expression patterns for neural and glial progenitor markers as assayed by RT-qPCR panel (FIG. 3B). Notably, bioreactor-generated OPCs exhibited strengthened extracellular contacts that led to differential enzymatic selections during dissociation. Therefore, comparison of stationary and agitation bioprocesses reveals several surprising and unexpected advantages of agitation including uniform sphere formation allowing for reduced variability in the culture and higher expression of key oligodendrocyte lineage markers as assayed by flow, immunofluorescence staining, and RT-qPCR. Agitation culture in bioreactors allows for higher initial seeding densities than stationary culture which enables higher yields at harvesting the cell product, allowing for further scalability to larger bioreactors. In some embodiments, the process is termed as a scalable differentiation platform.

### Dissociation of Oligospheres into Single Oligodendrocyte Progenitor Cells (OPCs)

The present disclosure describes a dissociation procedure which is scalable, time efficient with reduced operator handling and has improved batch-to-batch consistency.

The dissociation procedure combines enzymatic treatment and mechanical agitation. Additionally, the current method eliminates the need for exogenous DNase I during enzymatic treatment. Collectively, the current method results in high viable cell yield and post-thaw cell viability exceeding the benchmark of 70%.

In some of the embodiments, different types of agitators were tested, including but not limited to DASbox mini-bioreactor, Thermo Mixer C (Eppendorf) or Octo Dissociator (Miltenyi). All three provided tunability for rotational speed and heating (37° C.) capability.

In some of the embodiments, improved scalability relies on two components, source of enzymes and vessels used to perform the dissociation and both components needed to be scalable for this procedure to work.

In some embodiments, different types of enzyme mixtures/dissociation agents were employed including but not limited to Miltenyi Neurosphere Dissociation Kit (P), Accutase (1×), AccuMax, 10× TrypLE Select. Accutase followed by 2× TrypLE Select, 1× AccuMax plus 2× TrypLE Select or 4× TrypLE Select diluted in HBSS (Hanks' Balanced Salt Solution). In some embodiments, the enzymatic treatment comprises 1× AccuMax and 2× TrypLE Select diluted in HBSS or 4× TrypLE Select diluted in HBSS. The efficiency of enzymatic dissociation depends on the sufficient enzyme to cell mass ratio. In some embodiments, the enzyme to cell mass ratio is based on volume. For example, a sufficient ratio of cell (spheres):enzyme is 1:20 to 1:100. In some embodiments the ratio is 1:25 to 1:90, 1:30 to 1:80, 1:40 to 1:70, 1:50 to 1:60. In some embodiments, the DASbox mini-bioreactor allows enzyme supplies at scalable quantities. The DASbox mini-bioreactors hold up to 250 mL solution in each unit.

In some embodiments, the dissociation method using DASbox mini-bioreactors affords a significantly shortened time course. The mini-bioreactors of the disclosure enables a 3-hour procedure that can process up to about 800 mL or more of oligosphere culture. In some embodiments, the cell yield is defined as the number of viable single cells per milliliter of the original culture volume. The cell yield using DASbox mini-bioreactors in accordance with the present disclosure is about 1.3 millions/mL, about 1.35 millions/mL, about 1.4 millions/mL, about 1.45 millions/mL, about 1.5 millions/mL, about 1.55 millions/mL, about 1.59 millions/mL, about 1.60 millions/mL, about 1.65 millions/mL or about 1.69 millions/mL.

In some embodiments, the enzymatic treatment does not require addition of exogenous DNase I into the dissociation mixture. Conventionally, an issue upon dissociating any type of spheroids is the release of genomic DNA into the dissociation mixture, due to various degrees of compromised cell membrane or cell death in the process. As a result, the viscosity of the dissociating mixture is increased, and cell clumps may occur. Adding exogenous DNase I into the dissociation mixture is a common, art-recognized mitigation measure. However, the use of DNase I introduces foreign biological matter into the cell product. Complete elimination of DNase I after the procedure is either by dilution via extensive washing, which increases operator handling, or heat-inactivation at 65° C., which is incompatible with most cells. The method disclosed herein, has completely eliminated the need for DNase I.

In some embodiments, a lactate dehydrogenase release serves as a cell health indicator during sphere dissociation.

### Cryopreservation

Cryopreservation is a process that maintains biological samples in a state of suspended animation at cryogenic temperature for any considerable period and is used to preserve the fine structure of cells. The freezing behavior of the cells can be altered in the presence of a cryoprotective agent (also called cryoprotectant), which affects the rates of water transport, nucleation, and ice crystal growth. In some embodiments, the single oligodendrocyte progenitor cells (OPCs) were cryopreserved using a freezing process (control rate freezer) that is step-wise. The use of CRF (control rate freezer) ensures a batch-to-batch reproducibility. The type of cryoprotectant does not play an important role in and of itself. In some embodiments, the cryoprotectant is StemCell Banker. In some embodiments, the cryoprotectant is FresRS (FreSR™-S Single Cell Freezing Medium for ES/iPS Cells|STEMCELL Technologies) or BamBanker (BAMBANKER™ & BAMBANKER™ Direct|[Cell Culture] Products|Laboratory Chemicals—FUJIFILM Wako Chemicals U.S.A. Corporation).

### Treatment

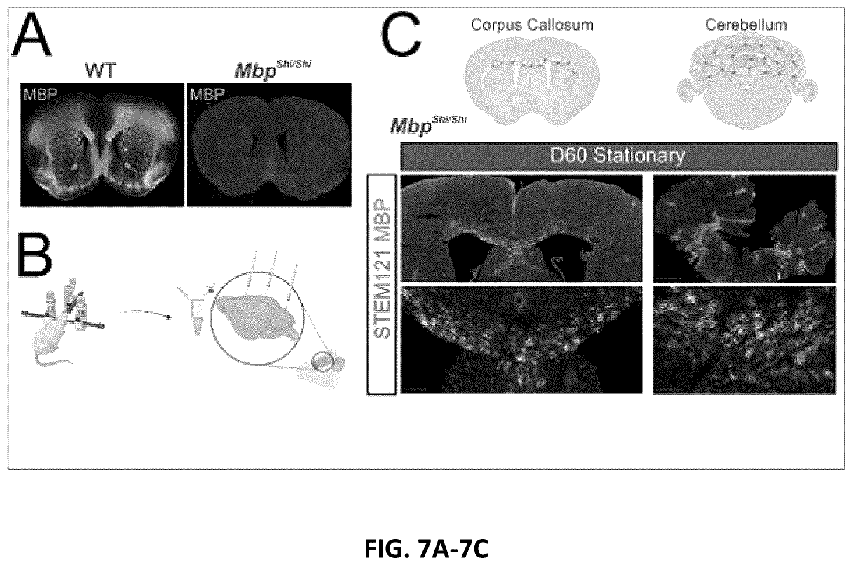

Disclosed herein are methods to treat demyelinating diseases and disorders using a cell replacement therapy approach. Demyelination describes a loss of myelin with relative preservation of axons. This results from diseases that damage myelin sheaths or the cells that form them. Oligodendrocyte progenitor cells (OPCs) are generated from induced pluripotent stem cells, derived from a healthy subject. PSC-derived oligodendrocyte progenitor cells (OPCs) are administered into the central nervous system of a subject to be treated. FIGS. 7A-7C show that, in accordance with the present disclosure, the PSC-derived OPCs successfully engrafted and matured into myelin basic protein (MBP)-expressing oligodendrocytes in the hypomyelinated mouse brain. Significantly, the mature oligodendrocytes successfully restored the expression of the myelin basic protein (MBP). The present disclosure is further illustrated by the following non-limiting examples.

### EXAMPLES

### Example 1

### Dissociation Protocol for Oligospheres

Neural progenitor cells (NPCs) were differentiated in a suspension culture in an impeller-driven DASbox mini-bioreactor for 60 days to generate oligospheres. The oligospheres were then settled for 30 minutes. The suspension culture medium was pumped out and dissociation reagent/enzyme mix (1× AccuMax and 2× TrypLE Select diluted in HBSS (Hanks' Balanced Salt Solution) or 4× TrypLE Select diluted in HBSS) was added. The dissociation reagent/enzyme mix did not include exogenous DNase I. The final bioreactor volume was 200 mL. Then the impeller of the DASbox mini-bioreactor was turned on to 100 rpm for 1 hour at 37° C. After 1 hour, the impeller speed was increased to 400 rpm for 2 hours at 37° C. 1 mL samples were taken every 30 minutes for cell counts and metabolite measurements. After 3 hours, the impeller was stopped when the oligospheres were dissociated into single oligodendrocyte progenitor cells (OPCs). Then the entire 200 mL cell suspension volume was taken out and passed through a 70 μm strainer and into a 250 mL centrifuge tube. Then it was centrifuged for 200 g×10 min. The supernatant was aspirated, and the pellet was resuspended in 40 mL E6 media supplemented with Y27632 (1:2000). If there were aggregates, it was passed through another 70 μm strainer. Then the final cell count was performed in 40 mL using 3 replicate 1 mL samples.

### Example 2

### Cryopreservation Protocol

After oligodendrocyte progenitor cells (OPCs) were dissociated into single cells, they were immediately resuspended into cryoprotectant solution, aliquoted, and slowly frozen to be stored in liquid nitrogen. After average cell count was recorded as described in Example 1, the cells were centrifuged for 200 g×10 min and resuspended in cryoprotectant solution (StemCell Banker) at either 5 million or 10 million/mL pre-chilled and labeled 1 mL cryotubes. These cryotubes/aliquots were brought on ice to control rate freezer (CRF) and the program was started. After the program was complete, the aliquots were transferred immediately to dry ice and then into liquid nitrogen tank storage. The CRF freezing program is a controlled, step-wise freezing process, designed specifically for mammalian cells, and batch-to-batch reproducibility. This program designs six steps to run samples from room temperature to −130° C. In some embodiments samples run from room temperature to −125° C. In some embodiments samples run from room temperature to −120° C. In some embodiments samples run from room temperature to −115° C. In some embodiments samples run from room temperature to −110° C. In some embodiments samples run from room temperature to −105° C. In some embodiments samples run from room temperature to −100° C. In some embodiments samples run from room temperature to −95° C. In some embodiments samples run from room temperature to −90° C. In some embodiments samples run from room temperature to −40° C. In some embodiments samples run from room temperature to −15° C. In some embodiments samples run from room temperature to −12° C. In some embodiments samples run from room temperature to −10° C. A key component is the first 10 minutes of rapid cooling to 0° C. This ensures every batch of cells reaches 0° C. within a tightly-controlled timeframe, and therefore, sample temperatures are synchronized before the next cooling phase begins.

### Example 3

### Mouse Ataxia Model

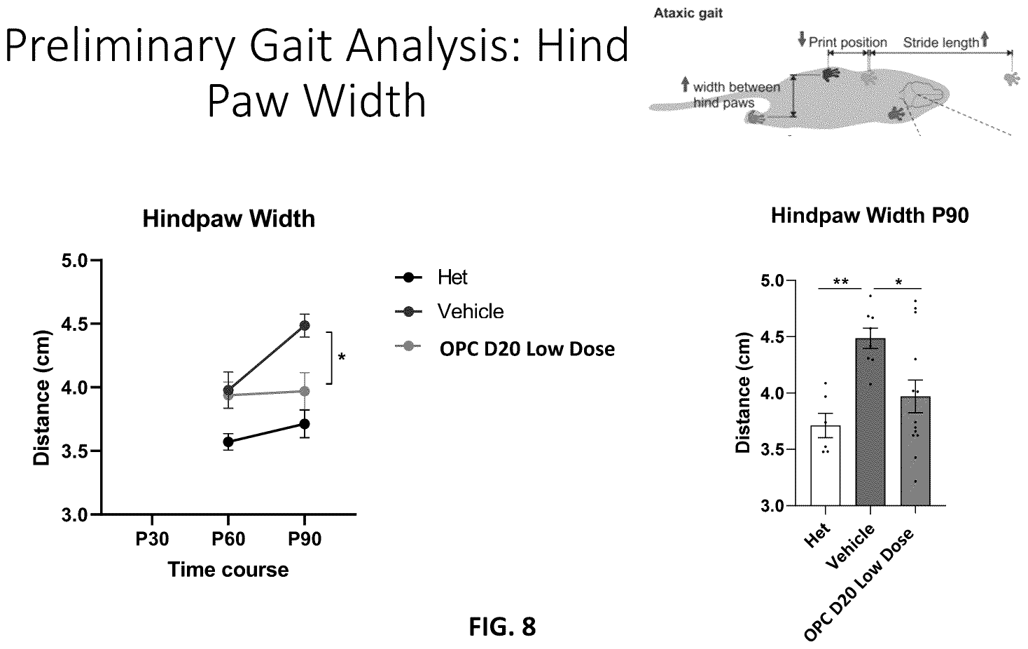

The efficacy of OPCs was assessed in the Shiverer mouse, a hypomyelinated mouse model. OPCs were transplanted into the brainstem and cerebellum (regions with large volumes of white matter, and importance for motor coordination) of Shiverer mice. OPCs rescued ataxic gait compared to vehicle controls (FIG. 8), a key phenotype observed in clinical populations. Furthermore, rescue in ataxic gait correlated with functional remyelination of the cerebellum as measured by the presence myelin basic protein (MBP) expression that is absent in the Shiverer mouse (data not shown). Conduction velocity was measured across major axon bundles, as a readout for functional myelination. Taken together, these data demonstrated that OPCs are sufficient to rescue molecular, functional, and subsequent behavioral phenotypes in a hypomyelinated mouse, suggesting that OPCs provide a treatment option for demyelinating disorders where existing therapeutics are limited.

What is claimed is:

1. A method of producing oligodendrocyte progenitor cells (OPCs) comprising:
obtaining induced pluripotent stem cells (iPSCs) from a subject; differentiating the iPSCs into neural progenitor cells (NPCs);
culturing the NPCs in a suspension culture in a bioreactor with agitation to form oligospheres;
dissociating the oligospheres using an enzymatic treatment and mechanical agitation into single oligodendrocyte progenitor cells (OPCs); and
cryopreserving the single OPCs in a step wise freezing process.

2. The method of claim 1, wherein the bioreactor is an impeller-driven DASbox mini-bioreactor.

3. The method of claim 2, wherein the DASbox mini-bioreactor has a speed of rotation of 100 rpm (rotation per minute) for about 1 hour followed by 400 rpm for about 2 hours.

4. The method of claim 1, wherein the agitation by the impeller-driven DASbox mini-bioreactor leads to high post-thaw cell viability after cryopreservation.

5. The method of claim 1, wherein the enzymatic treatment comprises 1× AccuMax and 2× TrypLE Select diluted in HBSS or 4× TrypLE Select diluted in HBSS (Hanks' Balanced Salt Solution).

6. The method of claim 1, wherein the enzymatic treatment does not include exogenous DNase I treatment.

7. The method of claim 1, wherein the NPCs are differentiated in the suspension culture for about 60 days.

8. The method of claim 1, wherein the OPCs express lineage markers CD9, O4, SOX10, OLIG2, and NKX2.2 by Day 60.

9. A method of treating a demyelinating disease comprising:
administering oligodendrocyte progenitor cells (OPCs) of claim 1 into a central nervous system of a subject to be treated;
allowing the administered oligodendrocyte progenitor cells (OPCs) to engraft in the central nervous system; and thereby
restoring the expression of a myelin basic protein (MBP).

10. A scalable differentiation platform to produce functional oligodendrocyte progenitor cells (OPCs), wherein the OPCs are produced in about 60 days in vitro.

11. The scalable differentiation platform of claim 10, wherein the OPCs are differentiated from neural progenitor cells (NPCs).

12. The scalable differentiation platform of claim 10, wherein the NPCs are differentiated in a suspension culture to form an oligosphere.

13. The scalable differentiation platform of claim 10, wherein the suspension culture is in an impeller-driven mini-bioreactors.

14. The scalable differentiation platform of claim 10, wherein the oligospheres generated in the impeller-driven mini-bioreactors allow for about a 4-fold expansion in cell yield and a physical uniformity of spheres.

15. The scalable differentiation platform of claim 10, wherein the OPCs express lineage markers CD9, O4, SOX10, OLIG2, and NKX2.2 by about day 60.

16. The scalable differentiation platform of claim 10, wherein a lactate dehydrogenase release serves as a cell health indicator during sphere dissociation.

17. The scalable differentiation platform of claim 10, wherein the scalable differentiation platform is a closed system.

18. An oligodendrocyte progenitor cell (OPC) produced by a method comprising:
obtaining induced pluripotent stem cells (iPSCs) from a subject;
differentiating the iPSCs into neural progenitor cells (NPCs);
culturing the NPCs in a suspension culture in a bioreactor with agitation to form oligospheres;
dissociating the oligospheres using an enzymatic treatment and mechanical agitation into single oligodendrocyte progenitor cells (OPCs); and
cryopreserving the single OPCs in a step wise freezing process.

19. Oligodendrocyte progenitor cells (OPCs), wherein the OPCs express lineage markers CD9, O4, SOX10, OLIG2, and NKX2.2 by about Day 60 after in vitro differentiation of neural progenitor cells (NPCs) in a suspension culture in a bioreactor.

20. The oligodendrocyte progenitor cells (OPCs) of claim 19, wherein said OPCs are modulated by WNT.

21. The oligodendrocyte progenitor cells (OPCs) of claim 19, wherein said OPCs are SOX10 and O4 positive at about Day 60.

22. The oligodendrocyte progenitor cells (OPCs) of claim 21, wherein the percentages of SOX10 and O4 positive cells at Day 60 increase to >50% with an addition of WNT modulator between about Day 40 to about Day 60.
